(19) United States
(12) Patent Application Publication
(10) Pub. No.: US 2004/0051052 A1
(43) Pub. Date: Mar. 18, 2004

(54) Method for manufacturing original disk for recording medium, and stamper manufacturing method

(76) Inventors: Shingo Imanishi, Kanagawa (JP); Asao Kurousu, Saitama (JP); Mitsuru Toyokawa, Tokyo (JP); Nobuyuki Arakawa, Kanagawa (JP)

Correspondence Address: ROBERT J. DEPKE LEWIS T. STEADMAN, HOLLAND & KNIGHT LLC, 131 SOUTH DEARBORN, 30TH FLOOR, CHICAGO, IL, 60603, US

(21) Appl. No.: 10/416,112

(22) PCT Filed: Sep. 3, 2002
(86) PCT No.: PCT/JP02/08942

(30) Foreign Application Priority Data
Sep. 5, 2001 (JP) 2001-26800

(51) Int. Cl. G21G 5/00; A61N 5/00

(52) U.S. Cl. 250/492.1

(57) ABSTRACT

The present invention is directed to a method of manufacturing an original plate used for manufacturing optical disc, which comprises: exposing and developing a photoresist layer provided on a base to form uneven pattern on the photoresist layer; irradiating ultraviolet rays to the photoresist layer where the uneven pattern is formed; and irradiating infrared rays onto the photoresist layer after ultraviolet rays have been irradiated thereto.

### TECHNICAL FIELD

The present invention relates to a method of manufacturing original plate used for manufacturing a recording medium, e.g., an optical recording medium, and a method of manufacturing stamper.

### BACKGROUND ART

Optical discs as optical recording medium adapted so that at least either one of audio data, video data and computer data, etc. is to be recorded, or has been recorded are widely used. As the optical disc, when roughly classified, there are optical disc of the reproduction (playback) only type and recordable disc. As the recordable type optical disc, there are write-once type optical disc which can record either one of the above-described data, but cannot carry out erasing (rewrite) operation, and erasable (rewrite) type optical disc which once records either one of the above-described data thereafter to have ability to carry out erasing (rewrite) operation.

These optical discs include a disc base (substrate) 201 having light transmission property such as polycarbonate, etc. as shown in FIG. 1, even if they are optical discs of either types of reproduction (playback) only type and recordable type. One surface of the disc base (substrate) 201 is caused to be a so called signal recording surface 201a, and the other surface is caused to be an incident surface 201b on which laser beams as light beams for recording or reproduction are incident.

In the case where the optical disc is recordable type optical disc, concentrical or spiral grooves 211 are formed from the inner circumferential side toward the outer circumferential side as shown in FIG. 2A at the signal recording surface 201a of the disc base (substrate) 201. In the case where the optical disc is the write-once type optical disc, recording layer consisting of organic pigmental material, reflection layer and protective layer are laminated in order so as to cover the grooves 211. In the case where the optical disc is erasable (rewrite) type optical disc, recording layer consisting of phase change optical recording material or magneto-optical recording material, reflection layer and protective layer are laminated in order so as to cover the grooves 211.

It is to be noted that track pitch P of the optical disc shown in FIG. 2A is distance from the falling wall portion of a certain groove 211 to the falling wall portion of the next groove 211.

In the case where the optical disc is optical disc of the reproduction only type, recording track consisting of plural pits 212 is formed as shown in FIG. 2B at the signal recording surface 201a of the disc base (substrate) 201 constituting this optical disc. This recording track is spirally formed from the inner circumferential side toward the outer circumferential side of the disc base (substrate) 201. Reflection layer and protective layer consisting of metallic material such as aluminum or gold, etc. are laminated so as to cover the pits 212 on the signal recording surface 201a of this disc base (substrate) 201 so that the optical disc of the reproduction only type is formed.

Read-out of data recorded in advance at the optical disc constituted as described above is carried out by irradiating laser beams for reproduction from the incident surface 201b side of the disc base 201 onto the signal recording surface 201a so that such laser beams are caused to be in focus state by object lens (objective) to detect laser beams reflected by the recording layer or the reflection layer. In order to record data onto the optical disc, laser beams for recording are irradiated from the incident surface 201b side of the disc base 201 onto at least the recording layer so that such laser beams are caused to be in focus state by object lens to record data with respect to at least either one of grooves 211 or so called land portions between grooves 211 along the grooves 211.

Meanwhile, with a view to realizing high recording density of data to be recorded onto optical disc, a method of shortening wavelength of laser beams emitted from semiconductor laser, which are used for recording data onto the optical disc or reproducing data recorded on the optical disc is being advanced. For example, when laser beams of short wavelength such as 410 nm or 405 nm, etc. like violet semiconductor laser are used, spot on the optical disc which has been converged by the object lens can be further reduced as compared to conventionally used laser beams of red or infrared semiconductor where wavelength is 780 nm or 650 nm, etc. Thus, spatial frequency characteristic is improved. Of course, in order to realize high recording density of optical disc, as shown in FIGS. 2A and 2B, this can be attained by shortening track pitch P, i.e., reducing track pitch P. However, reduction of track pitch P leads to physical limitation such as problem when disc base 201 is molded or formed, etc. Accordingly, attention is greatly drawn to realization of short wavelength of laser beams.

On the other hand, in the case where laser beams of short wavelength emitted from violet semiconductor laser are used to carry out recording of data onto the optical disc or to carry out reproduction of data recorded on the optical disc, it becomes apt to cope with also more fine structure of the disc surface. For example, by fine surface roughness of the signal recording surface 201a of the disc base 201 and/or molding (forming) distortion of the recording layer or the reflection layer at sharp twilled line portion constituted by uneven structure such as grooves 211 or pits 212 of the signal recording surface 201a, scattering, etc. takes place in reflected light of irradiated laser beams. For this reason, following realization of short wavelength of laser beams emitted from semiconductor laser, laser beams are scattered by fine surface roughness, etc. of the signal recording surface 201a at the time of recording data onto the optical disc, failing to sufficiently heat the recording layer. As a result, there is the possibility that correct data recording cannot be carried out.

In the case of carrying out reproduction of data recorded on the optical disc, noise component resulting from film formation distortion, etc. is increased in laser beams as reflected light reflected by the optical disc.

In order to reduce such noise component, it is sufficient to reduce fine surface roughness of the signal recording surface 201a of the disc base 201 to carry out chamfering of twilled line portion constituted by uneven structure such as groove 211, etc. For example, there is conceivable a method of irradiating ultraviolet rays onto the signal recording surface 201a of the disc base 201 consisting of synthetic resin to simultaneously realize smoothing of the surface of the signal recording surface 201a and curved surface of twilled line portion constituted by uneven structure such as groove 211, etc.

Since the time required for irradiating ultraviolet rays onto respective disc bases 201 one by one becomes order of “minute” in order to obtain valid effect by this method, productivity is very poor. The method of individually irradiating ultraviolet rays onto all disc bases 201 in the mass-production process of optical discs is not preferable in view of cost and time.

### DISCLOSURE OF THE INVENTION

An object of the present invention is to provide a method of manufacturing original plate used for manufacturing optical recording medium and a method of manufacturing stamper which can solve problems as described above that the optical disc has.

Another object of the present invention is to provide a method of manufacturing stamper and a method of manufacturing original plate used for manufacturing this stamper which can efficiently manufacture optical recording media adapted for carrying out recording of data and for carrying out reproduction of recorded data in the state where reduction of S/N (signal-to-noise ratio) is realized so that satisfactory recording/reproducing characteristic is obtained.

The present invention proposed in order to attain objects as described above is directed to a method of manufacturing original plate used for manufacturing optical recording medium, and this method comprises: exposing and developing a photoresist layer provided on a base; forming uneven pattern at the photoresist layer; irradiating ultraviolet rays onto the photoresist layer where the uneven pattern is formed; and thereafter irradiating infrared rays onto the photoresist layer.

In this method, plating processing is further implemented to the photoresist layer onto which infrared rays have been irradiated, whereby original plate is manufactured.

The present invention is directed to a method of manufacturing stamper for manufacturing an optical recording medium, which comprises: irradiating ultraviolet rays onto a ultraviolet hardening resin layer provide on a base and adapted so that uneven pattern is formed; and manufacturing a stamper by using the base including the ultraviolet hardening resin layer to which the ultraviolet rays have been irradiated.

The method of manufacturing stamper according to the present invention comprises: allowing a further base where a further ultraviolet rays hardening layer is provided to be in closely contact with the ultraviolet hardening resin layer to which the ultraviolet rays have been irradiated; irradiating ultraviolet rays to harden the further ultraviolet hardening resin layer to transfer the uneven pattern formed at the ultraviolet hardening resin layer to the further ultraviolet hardening resin layer; and implementing plating processing to the further ultraviolet hardening resin layer to which the uneven pattern has been transferred to form a stamper.

The present invention is directed to a further method of manufacturing stamper for manufacturing an optical recording medium, and this method comprises: exposing and developing a photoresist layer provided on an original plate to form uneven pattern at the photoresist layer; implementing plating processing to the photoresist layer where the uneven pattern has been formed to form master stamper; irradiating ultraviolet rays to a base where a ultraviolet hardening resin layer is provided and the master stamper in the state they are caused to be closely in contact with each other to transfer the uneven pattern of the master stamper to the ultraviolet hardening resin layer; thereafter further irradiating ultraviolet rays to the ultraviolet hardening resin layer; and manufacturing a stamper by using the base including the ultraviolet hardening resin layer to which the ultraviolet rays have been irradiated.

A further method of manufacturing stamper according to the present invention comprises: allowing a further base where a further ultraviolet hardening resin layer is provided to be in closely contact with ultraviolet hardening resin layer to which ultraviolet rays have been irradiated; irradiating ultraviolet rays to harden the further ultraviolet hardening resin layer to transfer the uneven pattern formed at the ultraviolet hardening resin layer to the further ultraviolet hardening resin layer; and implementing plating processing to the further ultraviolet hardening resin layer to which the uneven pattern has been transferred to form a stamper.

Still other objects of the present invention and practical merits obtained by the present invention will become more apparent from the description of the embodiments which will be given below with reference to the attached drawings.

### BRIEF DESCRIPTION OF THE DRAWINGS

FIG. 1 is a perspective view showing an example of an optical disc.

FIG. 2A is an essential part perspective view showing grooves provided at optical disc of the recordable type, and FIG. 2B is an essential part perspective view showing pits constituting recording track provided at an optical disc of the reproduction only type.

FIGS. 3A to 3D are cross sectional views showing, in step order, main steps of a method of manufacturing original plate of stamper for manufacturing optical recording medium and optical recording medium of a first embodiment according to the present invention.

FIGS. 4A to 4D are cross sectional views showing, in step order, further steps of the method of manufacturing original plate of stamper for manufacturing optical recording medium and optical recording medium of the first embodiment according to the present invention.

FIG. 5 is a view showing irradiating intensity at ultraviolet rays irradiation used in the method of manufacturing original plate of stamper for manufacturing optical recording medium according to the present invention.

FIGS. 6A to 6E are cross sectional views showing, in step order, main steps of a method of manufacturing original plate of stamper for manufacturing optical recording medium and optical recording medium of a second embodiment according to the present invention.

FIGS. 7A to 7C are cross sectional views showing, in step order, further steps of the method of manufacturing original plate of stamper for manufacturing optical recording medium and optical recording medium of a second embodiment according to the present invention.

FIGS. 8A to 8D are cross sectional views showing, in step order, main steps of a method of manufacturing original plate of stamper for manufacturing optical recording medium and optical recording medium of a third embodiment according to the present invention.

FIGS. 9A to 9C are cross sectional views showing, in step order, further steps of the method of manufacturing original plate of stamper for manufacturing optical recording medium and optical recording medium of the third embodiment according to the present invention.

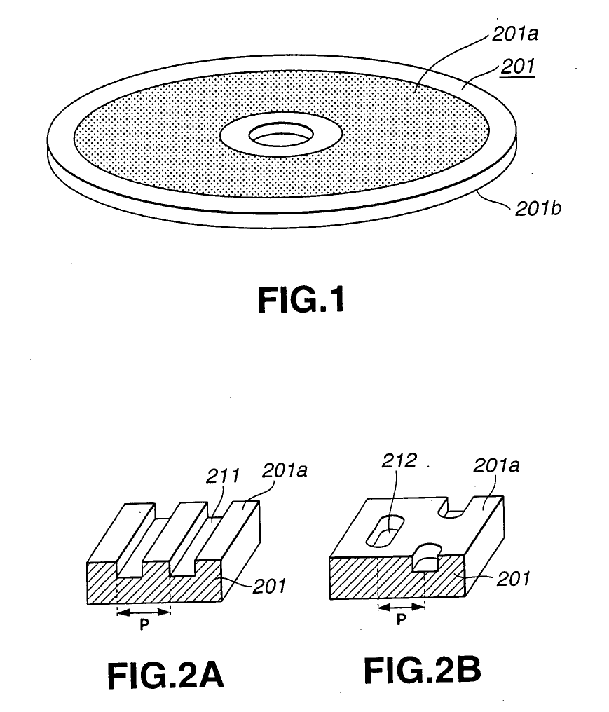

### BEST MODE FOR CARRYING OUT THE INVENTION

Embodiments of the present invention will now be described in detail with reference to the attached drawings.

First Embodiment

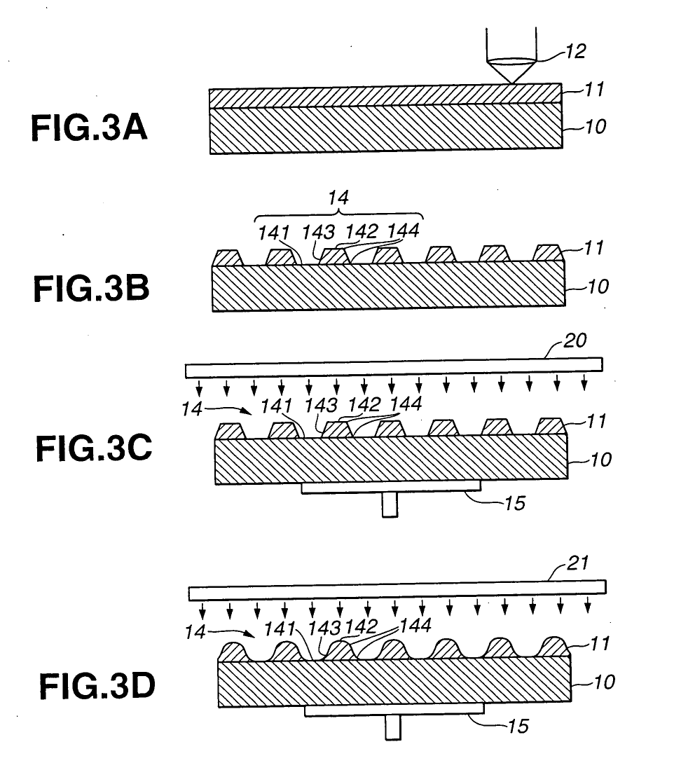

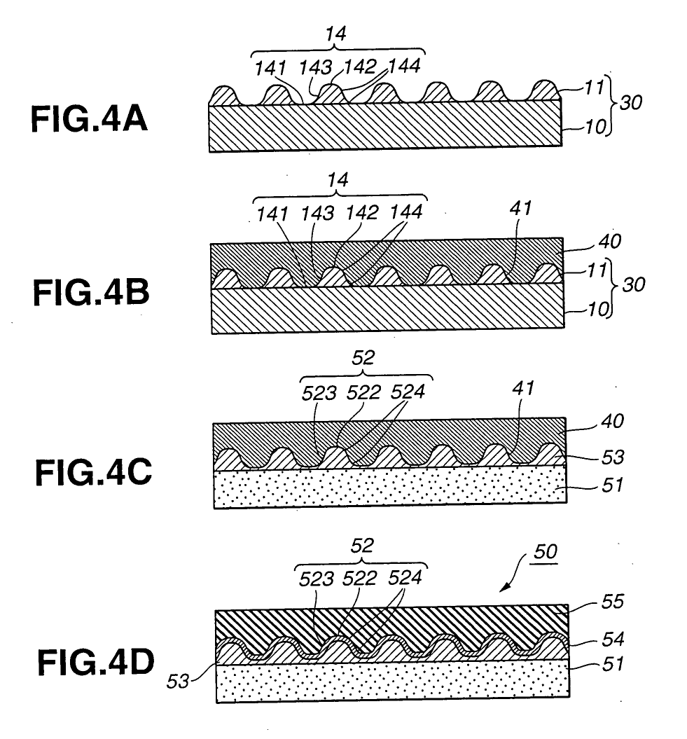

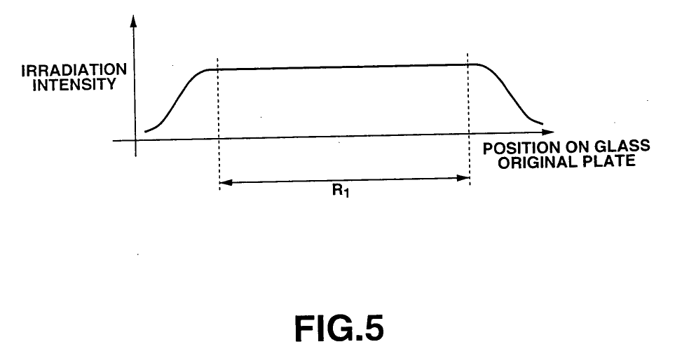

FIGS. 3A to 3D are cross sectional views showing steps of manufacturing an original plate of a stamper for manufacturing optical recording medium by the first embodiment of the present invention, and FIGS. 4A to 4D are cross sectional views showing steps of manufacturing optical disc which is optical recording medium.

In order to manufacture an original plate used for manufacturing a stamper by the method of the present invention, as shown in FIG. 3A, e.g., a glass original plate 10 polished and rinsed so that the surface thereof is flat is mounted on rotational stage (not shown) to rotate this glass original plate 10 under the condition where, e.g., linear velocity is constant. On this rotating glass original plate 10, e.g., photoresist 11 of the so-called positive type which is photosensitive material layer which becomes alkali soluble, e.g., by carrying out sensitizing is coated so that it has uniform thickness of, e.g., about 100 nm. In this case, this photoresist 11 may be of negative type. For example, there may be employed resist of any kind such as novolak system resist or chemical amplification type resist, etc. It is sufficient that film thickness of this photoresist 11 is about 30˜150 nm.

Then, the glass original plate 10 on which the photoresist 11 is coated is rotated to converge laser beams which has been caused to undergo intensity modulation in correspondence with recording signal onto the photoresist 11 by an object lens (objective) 12 to expose the photoresist 11. At this time, positions of the glass original plate 10 and the object lens 12 are caused to relatively undergo parallel displacement at a predetermined velocity in a radial direction of the glass original plate 10 to thereby form latent image of an uneven pattern 14 in a spiral form which will be described later. For example, at least the object lens 12 is caused to undergo parallel displacement at a predetermined velocity in a radial direction of the glass original plate 10 while rotating the glass original plate 10 to irradiate intensity-modulated laser beams onto the photoresist 11 to thereby form latent image. Without limiting to this, there may be also employed an approach to move the glass original plate 10 at a predetermined velocity also in the radial direction while rotating it at a predetermined number of revolutions in the state where the object lens 12 is not moved to thereby form latent image at the photoresist 11.

When the region sensitized by laser beams is dissolved by developing photoresist 11 exposed in a manner as described above by, e.g., alkali developer, uneven pattern 14 corresponding to at least either one of grooves and pits recorded onto the optical disc is formed as shown in FIG. 3B. For example, laser beams irradiated onto the photoresist 11 are intermittently turned ON and OFF on the basis of data to be recorded, whereby uneven pattern 14 corresponding to pits shown in FIG. 2B is formed. Laser beams are continuously irradiated onto the photoresist 11, whereby uneven pattern 14 corresponding to grooves shown in FIG. 2A is formed. This uneven pattern 14 consists of a glass flat surface portion 141 which is the surface exposed as the result of the fact that the photoresist 11 on the original plate 10 is dissolved, and a photoresist flat surface portion 142 which is the remaining surface which is not dissolved by development processing because it is not sensitized on the photoresist 11. The remaining portion which is not dissolved on the photoresist 11 forms an inclined surface portion 143 as shown in FIG. 3B. Twilled line portions 144 are formed at the boundary between the photoresist flat surface portion 142 and the inclined surface portion 143 and the boundary between the glass flat surface portion 141 and the inclined surface portion 143.

Ultraviolet ray irradiation and infrared ray irradiation are implemented onto the surface of the photoresist 11 where the uneven pattern is formed on the glass original plate 10 in this way in a manner as described below. Thus, an original plate 30 for duplication of stamper which is an original plate of stamper for manufacturing optical recording medium is made or produced.

Namely, as shown in FIG. 3C, the surface opposite to the surface where the uneven pattern 14 is formed of the glass original plate 10 is attached to a rotary stage 15 to rotate the glass original plate 10 at a constant velocity, e.g., about 60 rpm. Onto the surface of the photoresist 11 where the uneven pattern 14 is formed on the rotated glass original plate 10, ultraviolet rays are irradiated, e.g., for about four minutes and at the intensity of about 10 mW/cm² by plural ultraviolet lamps 20 arranged in parallel to each other under existence of oxygen or air. These ultraviolet lamps 20 are disposed in the state where they are in parallel to the surface of the photoresist 11 where the uneven pattern 14 is formed on the glass original plate 10, and height from the surface of the photoresist 11 is caused to be constant, e.g., about 100 mm. The glass original plate 10 is rotated at a fixed velocity by the rotary stage 15 and height from the glass original plate 10 of the ultraviolet lamp 20 is caused to be constant, whereby irradiation intensity of ultraviolet rays substantially becomes constant and irradiation intensity surface density of ultraviolet rays also substantially becomes constant within the effective size R₁ of the surface where the uneven pattern 14 is formed of the glass original plate 10. In this example, as the ultraviolet lamp 20, e.g., excimer lamp having wavelength of 174 nm or low pressure mercury lamp, etc. may be used. In place of rotating the glass original plate 10 by using the rotary stage 15, ultraviolet lamp 20 may be rotated at a constant velocity.

The above-described respective conditions are values determined by the result of the experiment. For example, when product of intensity of ultraviolet rays and irradiation time thereof becomes great, roughness of the surface of the photoresist 11 becomes great and thickness of the photoresist 11 becomes thin. Conversely, when that product becomes small, there takes place the problem that realization of curved surface of the twilled line portion which will be described later of the photoresist 11 is not sufficiently carried out, etc. The intensity and the irradiation time of ultraviolet rays are not limited to those of the above-described example, but may be suitably selected in accordance with material of the photoresist 11.

When ultraviolet rays are irradiated from the ultraviolet lamps 20 under existence of oxygen or air onto the surface of the photoresist 11 where the uneven pattern 14 is formed on the glass original plate 10, ultraviolet rays are first irradiated to oxygen (O₂) so that ozone (O₃) is produced. Since this ozone (O₃) is unstable, it is decomposed into oxygen (O₂) and excited oxygen (O*) having very strong oxidation power. This excited oxygen (O*) oxidizes the surface of the photoresist 11 which is organic matter to reform it. Thus, as shown in FIG. 3D, the twilled line portion 144 of the uneven pattern 14 is chamfered and is curved.

On the other hand, at the same time when the twilled line portion 144 of the uneven pattern 14 is rounded, roughness of the surface of the photoresist 11 including the photoresist flat surface portion 142 and the inclined surface portion 143 constituting the uneven pattern 14 is slightly increased. In view of this, the ultraviolet lamps 20 are replaced by plural infrared lamps 21 arranged in parallel to each other. By these infrared lamps 21, infrared rays are irradiated, e.g., for about three minutes and at the intensity of about 20 W/cm² onto the surface where the uneven pattern 14 is formed of the glass original plate 10 rotated at a constant velocity by the rotary stage 15 subsequently to irradiation of ultraviolet rays. As this infrared lamp 21, e.g. halogen lamp is used. It is preferable that surface arrival temperature with respect to the glass original plate 10 by the infrared lamp 21 is more than about 200° C. although it is varied in dependency upon wavelength of infrared rays. In the case where, e.g., near infrared rays (visible light˜wavelength about 5 μm) is used, it is preferable that the surface arrival temperature is about 250° C. In the case where far infrared rays (wavelength about 1˜8 μm) is used, it is preferable that the surface arrival temperature is about 400° C. The reason why it is preferable that the surface arrival temperature is more than about 200° C. is that it is necessary to heat the photoresist 11 so that temperature reaches more than softening point in order to carry out smoothing of the surface of the photoresist 11 by irradiation of infrared rays as described later. It is to be noted that the reason why arrival temperatures by near infrared rays and far infrared rays are different is that absorption factor with respect to photoresist is varied by wavelength of infrared rays.

The above-described irradiation time and irradiation intensity of infrared rays are based on the result of the experiment. When product of the irradiation time and the irradiation intensity becomes great, the uneven pattern 14 itself of the photoresist 11 is deformed and/or the photoresist 11 is carbonized in a manner exceeding smoothing of the photoresist 11. Conversely, when product of the irradiation time and the irradiation intensity becomes small, smoothing of the surface of the photoresist 11 cannot be sufficiently carried out.

It is to be noted that the fact that the glass original plate 10 is rotated at a constant velocity on the rotary stage 15 and the infrared lamp 21 is disposed at a predetermined height from the glass original plate 10 so that irradiation intensity surface density within the effective size of the surface where the uneven pattern 14 is formed of the glass original plate 10 substantially becomes constant is similar to the case of the above-described ultraviolet irradiation. By irradiation of infrared rays onto the surface where the uneven patten 14 is formed of this glass original plate 10, as shown in FIG. 4A, the surface of the photoresist 11 including the photoresist flat surface portion 142 and the inclined surface portion 143 constituting the uneven pattern 14 is softened as the result of the fact that temperature rises, and becomes smooth by surface tension. As a result, there can be obtained the original plate 30 for duplication of stamper in which the surfaces of the photoresist flat surface portion 142 and the photoresist inclined surface portion 143 constituting the uneven pattern 14 are smoothed, and the twilled line portion 144 of the uneven pattern 14 is curved.

In the above-described example, in order to carry out ultraviolet irradiation and infrared irradiation with respect to the glass original plate 10, ultraviolet lamps 20 and infrared lamps 21 were used. In place of these lamps, ultraviolet irradiation unit and infrared irradiation unit may be respectively used.

As shown in FIG. 4B, electrically conductive film (not shown) is formed by, e.g., electroless plating method on the surface where the uneven pattern 14 is formed of the original plate 30 for duplication of stamper obtained in this way thereafter to implement, e.g., Ni (nickel) plating by electroplating method to form a Ni plating layer 40. This Ni plating layer 40 is peeled off from the original plate 30 for duplication of stamper. As a result, the uneven pattern 14 of the original plate 30 for duplication of stamper is transferred to the Ni plating layer 40 which has been peeled off. Thus, there results stamper 40 in which the uneven pattern 41 has been formed. It is to be noted that organic matter such as photoresist 11, etc. reformed by the above-described irradiation of ultraviolet rays, etc. is attached on the surface where the uneven pattern 41 is formed of the Ni plating layer 40, i.e., the stamper 40. For this reason, e.g., it is necessary to rinse, by organic solvent, the surface where the uneven pattern 41 has been formed of the stamper 40 thereafter to irradiate deep ultraviolet rays to dissolve and rinse organic matter such as photoresist 11, etc.

Then, as shown in FIG. 4C, this stamper 40 is used to press, by a predetermined pressure, the stamper 40 to ultraviolet hardening resin coated on a disc base 51 consisting of flat transparent resin, e.g., glass, polymethyl methacrylate (PMMA) or polycarbonate (PC), etc. by, e.g., Photo Polymerization method (2P method) to irradiate ultraviolet rays from the other surface of the disc base in that state to harden it, whereby a transfer layer 53 where uneven pattern 41 has been transferred of the stamper 40 is formed. Thus, an uneven pattern 52 comprised of train of grooves and/or pits is formed at the disc base 51 which is a base for optical recording medium. It is to be noted that, in addition to the 2P method, stamper 40 may be attached to metal mold to form uneven pattern 52 at one surface of the disc base 51 by injection molding or compression molding by using polymethyl methacrylate (PMMA) or polycarbonate (PC).

As shown in FIG. 4D, on the transfer layer 53 of the disc base 51 where the uneven pattern 52 has been formed, a recording layer 54 including phase change film or magnetic film, etc. consisting of, e.g., recordable optical recording material of the phase change type or magneto-optical recording material and a protective layer 55 are formed in order. In practical sense, e.g., dielectric film consisting of SiN, etc., perpendicular magnetic recording film consisting of TeFeCo alloy, etc., dielectric film consisting of SiN, etc. and reflection film consisting of Al, etc. are laminated in order by the sputtering method on the surface where the uneven pattern 52 is formed to thereby form the recording layer 54. Thereafter, ultraviolet hardening resin is coated on this recording layer 54 by the spin-coat method to irradiate ultraviolet rays onto the coated ultraviolet hardening resin to harden it so that the protective layer 55 is formed. Thus, optical disc 50 which is optical recording medium can be obtained. This optical disc 50 is not limited to magneto-optical disc or phase change type optical disc, but may be, e.g., reproduction only optical disc or optical card, etc. In the case of the reproduction only optical disc, it is sufficient to form light reflection layer consisting of, e.g., Al, etc. and protective layer consisting of, e.g., ultraviolet hardening resin, etc. on this light reflection layer.

While recording layer 54 and protective layer 55 are formed on transfer layer 53 in the above-described example, when uneven pattern 52 is formed on one surface of disc base 51, recording layer 54 and protective layer 55 may be formed in order on one surface of base 51.

As stated above, in this embodiment, ultraviolet rays and infrared rays are irradiated at a uniform intensity onto the surface of the photoresist 11 where the uneven pattern 14 has been formed on the glass original plate 10, thereby making it possible to manufacture original plate 30 for duplication of stamper in which twilled line portion 144 of uneven pattern 14 has been curved and the surfaces of the photoresist flat surface portion 142 and the inclined surface portion 143 have been smoothed. This original plate 30 for duplication of stamper is used to transfer uneven pattern 14 onto stamper 40, thus making it possible to form, at the disc base 51 manufactured by using this stamper 40, uneven pattern 52 including a flat portion 522 and an inclined surface portion 523 of which surfaces have been smoothed, and a twilled line portion 524 which has been curved. In the optical disc 50 including this optical disc base 51, it can be prevented that surface roughness takes place at the surface where the uneven pattern 52 is formed of the disc base 51, and/or distortion in film formation takes place in recording layer 54, etc. at the twilled line portion 524 of the uneven pattern 52.

As described above, even in the case where laser beams of short wavelength like, e.g., violet semiconductor laser are irradiated onto this optical disc 50 to carry out recording or reproduction of information, noise can be reduced so that sufficient S/N can be ensured. It becomes unnecessary to irradiate ultraviolet rays onto respective disc bases one by one as previously described above to manufacture such optical discs. In the above-described embodiment, stamper 40 in which uneven pattern 14 has been transferred from original plate 30 for duplication of stamper to which ultraviolet rays and infrared rays have been irradiated is used, thereby making it possible to efficiently mass-produce disc bases and optical discs at low cost and in a short time.

Second Embodiment

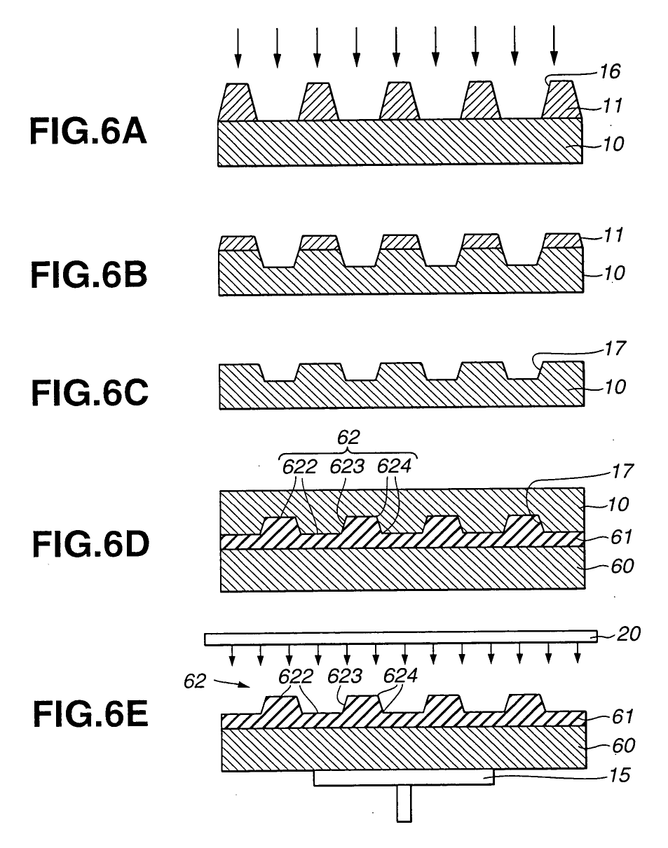

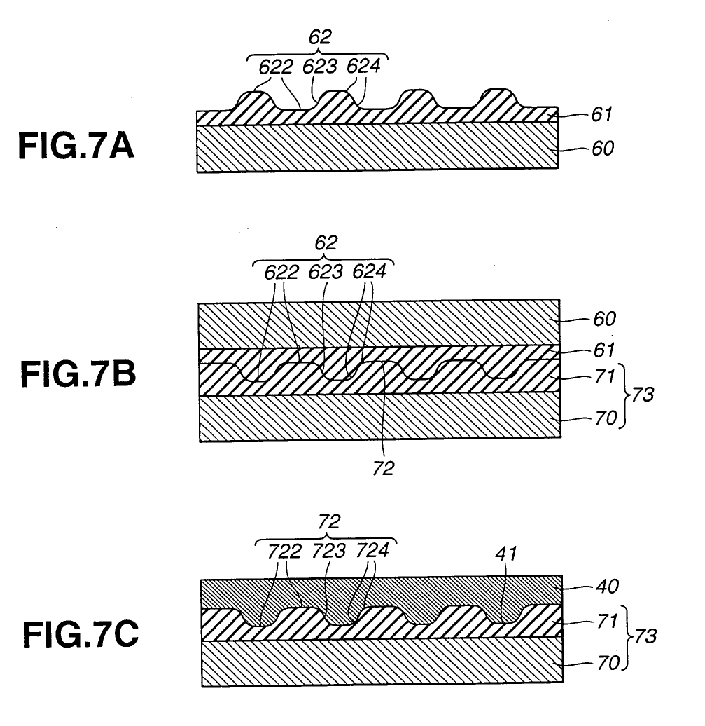

Then, a method of manufacturing original plate of a stamper for manufacturing optical recording medium and a method of manufacturing stamper which are a second embodiment according to the present invention will be explained with reference to FIGS. 6A to 6E and FIGS. 7A to 7C. In the second embodiment, steps up to ones for manufacturing optical recording medium from stamper are similar to those of the first embodiment. Accordingly, explanation will be given below only in connection with points different from the first embodiment, and the explanation of the first embodiment is invoked (applied) in connection with other points.

First, as shown in FIG. 6A, photoresist 11 coated on glass original plate 10 so as to have uniform thickness of about 200˜300 nm is exposed and developed similarly to the first embodiment to thereby form uneven pattern 16.

Anisotropic etching is implemented to the glass original plate 10 where the uneven pattern 16 has been formed by, e.g., Reactive Ion Etching (RIE). As shown in FIG. 6B, a portion of the photoresist 11 which has been left by development is removed by this Reactive Ion Etching, and the glass original plate 10 of the portion exposed as the result of the fact that the photoresist 11 is removed by development is dug down. The quantity in which the glass original plate 10 is dug down is caused to be, e.g., about 100 nm. As described above, by thickening coating film of the photoresist 11 so that its thickness becomes equal to about 200˜300 nm, or setting kind of etching gas or chamber internal pressure, etc. so that etching rate, i.e., ratio between height in which the glass original plate 10 is dug down and height in which the photoresist 11 is dug down becomes great, it can be prevented that even the glass original plate 10 under the photoresist 11 left by development is also dug down.

Then, as shown in FIG. 6C, the photresist 11 left on the surface of the glass original plate 10 after Reactive Ion Etching is removed by rinsing it with organic solvent, or by irradiating deep ultraviolet light thereto, etc. Thus, on the glass original plate 10 from which the photoresist 11 has been removed, an uneven pattern 17 corresponding to grooves and/or pits is formed. It is to be noted that there can be also obtained the effect or advantage that the surface of the uneven pattern 17 on the glass original plate 10 is smoothed by this Reactive Ion Etching.

Then, as shown in FIG. 6D, the surface of the glass original plate 10 is sufficiently rinsed by ultrasonic pure water cleaning thereafter to transfer the uneven pattern 17 of the glass original plate 10 to, e.g., an acrylic original plate 60 by the 2P method with this glass original plate 10 being as matrix. Namely, ultraviolet hardening resin is first coated onto the glass original plate 10 where the uneven pattern 17 has been formed to allow the acrylic original plate 60 to be closely stuck onto this ultraviolet hardening resin layer 61. After ultraviolet rays are irradiated from either the glass original plate 60 or the acrylic original plate 60 to harden ultraviolet hardening resin layer 61, the glass original plate 10 is peeled off from the ultraviolet hardening resin layer 61. Thus, the uneven pattern 17 of the glass original plate 10 is transferred to the ultraviolet hardening resin layer 61. Thus, the acrylic original plate 60 including the ultraviolet hardening resin layer 61 where the uneven pattern 62 has been formed is produced. This uneven pattern 62 is composed of a flat surface portion 622, an inclined surface portion 623, and a twilled line portion 624. It is to be noted that glass original plate 10 which has been caused to undergo anisotropic etching can be used many times for manufacturing of the acrylic original plate 60 by the 2P method as matrix.

Then, as shown in FIG. 6E, the surface opposite to the surface where the uneven pattern 62 has been formed of the acrylic original plate 60 is supported by the rotary stage 15 to rotate this rotary stage 15 at a constant velocity, e.g., about 60 rpm. Onto the surface of the hardened ultraviolet hardening resin layer 61 where the uneven pattern 62 has been formed on the rotated acrylic original plate 60, ultraviolet rays are irradiated, e.g., for about four minutes and at an intensity of about 10 mW/cm² by plural ultraviolet lamps 20 arranged in parallel to each other under the existence of oxygen or air. The irradiation time and the irradiation intensity of ultraviolet rays are based on result of the experiment, and time and intensity are suitably selected in accordance with composition of material of ultraviolet hardening resin and to what degree the uneven pattern 62 is curved. These ultraviolet lamps 20 are adapted so that height from the surface of the ultraviolet hardening resin layer 61 where the uneven pattern 62 has been formed of the acrylic original plate 60 on the rotary stage 15 is caused to be constant, whereby irradiation intensity surface density within the effective size of the surface where the uneven pattern 62 has been formed of the acrylic original plate 60 substantially becomes constant similarly to the previously described first embodiment. As the ultraviolet lamp 20, e.g., excimer lamp having wavelength of 174 nm or low pressure mercury lamp, etc. may be used. In place of rotating the acrylic original plate 60 by using the rotary stage 15, ultraviolet lamp 20 may be rotated at a constant velocity similarly to the first embodiment.

Ultraviolet rays are irradiated from the ultraviolet lamps 20 onto the surface of the ultraviolet hardening resin layer 61 where the uneven pattern 62 has been formed on this acrylic original plate 60 under existence of oxygen or air, and ultraviolet rays are first irradiated to oxygen (O₂) so that ozone (O₃) is produced. Since this ozone (O₃) is unstable, it is decomposed into oxygen (O₂) and excited oxygen (O*) having very strong oxidation power. This excited oxygen (O*) oxidizes the surface of the ultraviolet hardening layer 61 which is organic matter to reform it. Thus, as shown in FIG. 7A, the twilled line portion 624 of the uneven pattern 62 is chamfered and is curved, and the surface of the ultraviolet hardening resin layer 61 including the flat surface portion 622 and the inclined surface portion 623 constituting the uneven pattern 62 is smoothed at the same time.

It is to be noted that while there were cases in the first embodiment such that ultraviolet rays are irradiated onto the photoresist 11, whereby roughness of the surface thereof is slightly increased, there is no possibility in the second embodiment that even if ultraviolet rays are irradiated onto the hardened ultraviolet hardening resin layer 61, roughness of the surface is increased, but the surface becomes more smooth. Accordingly, in the second embodiment, there is no necessity of irradiating infrared rays as in the case of the first embodiment. It should be noted that while plural ultraviolet lamps arranged in parallel to each other were used for the purpose of carrying out ultraviolet rays irradiation with respect to the acrylic original plate 60 in the above-described second embodiment, an ultraviolet irradiation unit may be used in place of these ultraviolet lamps.

Then, as shown in FIG. 7B, with this acrylic original plate 60 to which ultraviolet rays have been irradiated being as matrix, the uneven pattern 62 of the acrylic original plate 60 is transferred to a polished glass original plate 70 by the 2P method. Namely, first, ultraviolet hardening resin is further coated onto the ultraviolet hardening resin layer 61 where the uneven pattern 62 has been formed on the acrylic original plate 60 to closely stick the glass original plate 70 onto this coated ultraviolet hardening resin layer 71. Of course, ultraviolet hardening resin may be coated onto glass original plate 70 to closely stick acrylic original plate 60 thereto. Ultraviolet rays are irradiated from either the acrylic original plate 60 or the glass original plate 70 to harden ultraviolet hardening resin layer 71 thereafter to peel off the acrylic original plate 60 from the ultraviolet hardening resin layer 71. Thus, there is produced or manufactured an original plate 73 for duplication of stamper including the ultraviolet hardening resin layer 71 in which there has been formed an even pattern 72 to which the uneven pattern 62 of the acrylic original plate 60 has been transferred. The step of transferring uneven pattern 62 to the glass original plate 70 by the 2P method with this acrylic original plate 60 being as matrix is a step provided for the reason why it is difficult to implement Ni plating to the surface where the uneven pattern 62 has been formed at the ultraviolet hardening resin layer 61 on the acrylic original plate 60 because the surface of the ultraviolet hardening resin layer 61 to which ultraviolet rays have been irradiated is reformed. It is to be noted that acrylic original plate 60 to which ultraviolet rays have been irradiated can be used many times for manufacturing of glass original plate 70 by the 2P method as matrix.

Then, as shown in FIG. 7C, electrically conductive film (not shown) is formed by, e.g., electroless plating method on the surface where the uneven pattern 72 has been formed at the ultraviolet hardening resin layer 71 on the original plate 73 for duplication of stamper thereafter to implement, e.g., Ni plating by the elctroplating method to form Ni plating layer 40. By peeling off this Ni plating layer 40 from the original plate 73 for duplication of stamper, the Ni plating layer 40 which has been peeled off results in the stamper 40 in which the uneven pattern where there has been formed the uneven pattern 72 of the original plate 73 for duplication of stamper has been transferred. It is to be noted that the original plate 73 for duplication of a stamper can be used many times for manufacturing of the stamper 40 as matrix. Steps subsequent to the step of transferring uneven pattern 41 from this stamper 40 onto disc base 51 are carried out similarly to the first embodiment.

As stated above, in the second embodiment, by irradiating ultraviolet rays at a uniform intensity onto the ultraviolet hardening resin layer 61 where uneven pattern 62 has been formed, it is possible to manufacture acrylic original plate 60 in which the surfaces of the flat surface portion 622 and the inclined surface portion 623 constituting the uneven pattern 62 have been smoothed, and the twilled line portion 624 of the uneven pattern 62 has been curved. By using this acrylic original plate 60, it is possible to manufacture an original plate 73 for duplication of stamper in which the surfaces of a flat surface portion 722 and an inclined surface portion 723 of the uneven pattern 72 have been smoothed, and a twilled line portion 724 of the uneven pattern 72 has been curved. As disc base 51 manufactured by using the stamper 40 manufactured on the basis of this original plate for duplication of stamper, it is possible to form uneven pattern 52 including flat surface portion 522 and inclined surface portion 523 of which surfaces have been smoothed and twilled line portion 524 which has been curved. Thus, in the optical disc 50 including this disc base 51, it can be prevented that surface roughness takes place at the surface where uneven pattern 52 is formed of the disc base 51, and/or distortion in film formation takes place in the recording layer 54, etc. at the twilled line portion 524 of the uneven pattern 52.

As described above, even in the case where laser beams of short wavelength like, e.g., violet semiconductor laser are irradiated onto this optical disc 50 to carry out recording of information or to carry out reproduction of recorded information, noise can be reduced, and sufficient S/N can be ensured. In addition, it is possible to efficiently mass-produce such optical discs 50 at low cost and in a short time by using stamper 40 including uneven pattern 41 based on uneven pattern 62 of acrylic original plate 60.

Since this glass original plate 10 which has been caused to undergo anisotropic etching can be used many times for manufacturing of acrylic original plate 60 by the 2P method as matrix, it is possible to produce, at low cost and in a short time, acrylic original plate 60 including ultraviolet hardening resin layer 61 where uneven pattern 62 has been formed.

Further, since the acrylic original plate 60 can be used many times for manufacturing of glass original plate 70 by the 2P method as matrix, it is possible to produce original plate 73 for duplication of stamper including ultraviolet hardening resin layer 71 where uneven pattern 72 has been formed.

Since the original plate 73 for duplication of stamper can be used many times for manufacturing of the stamper 40 as matrix, it is possible to produce, at a low cost and in a short time, stamper 40 in which uneven pattern 41 has been formed.

Third Embodiment

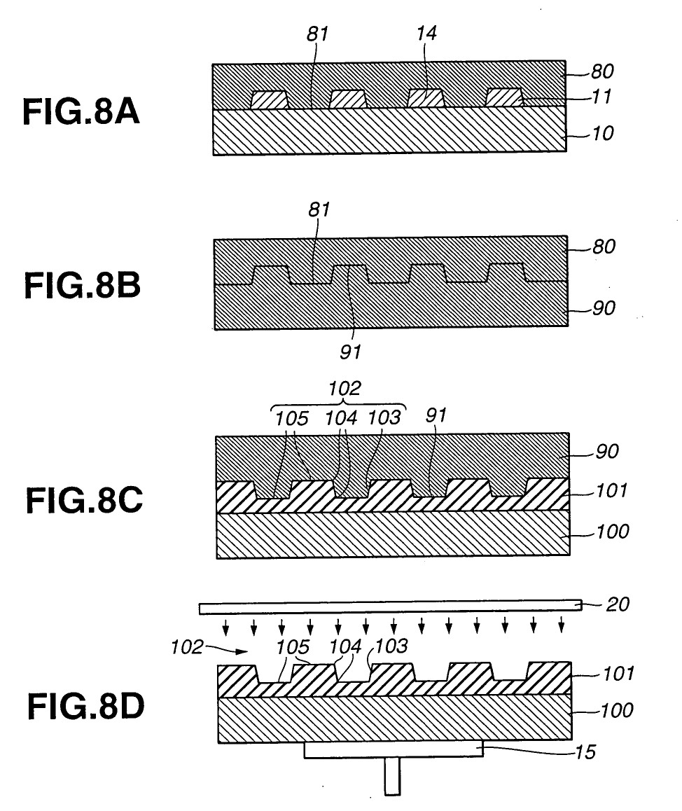

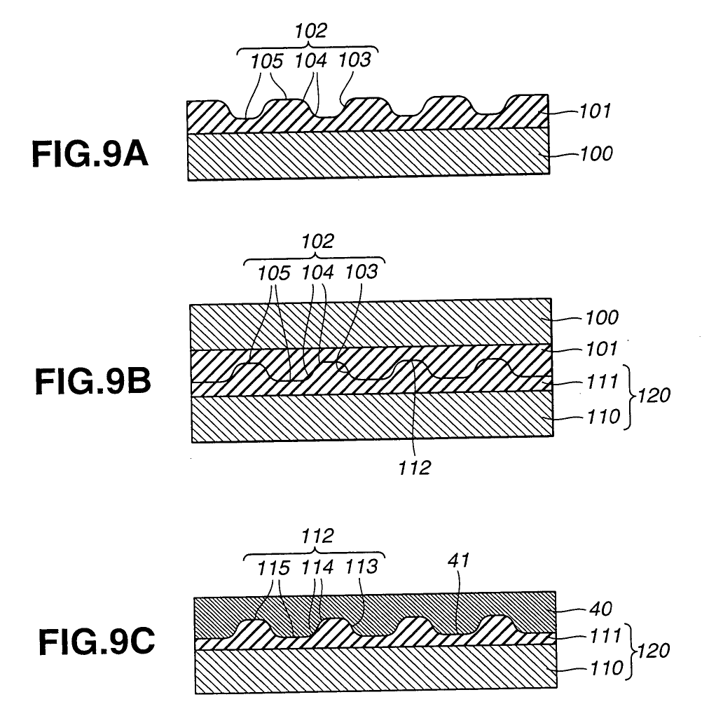

Then, a method of manufacturing original plate of stamper for manufacturing optical recording medium and a method of manufacturing stamper which are the third embodiment according to the present invention will be explained with reference to FIGS. 8A to 8D and FIGS. 9A to 9C. In the third embodiment, steps up to ones of forming uneven pattern at photoresist on glass original plate, and steps up to ones of manufacturing optical disc from stamper are similar to those of the first embodiment. Accordingly, explanation will be given below only in connection with the points different from the first embodiment, and explanation of the first embodiment is invoked or applied in connection with other points.

First, as shown in FIG. 8A, photoresist 11 coated on glass original plate 10 is exposed and developed so that electrically conductive film (not shown) is formed by, e.g., electroless plating method on the surface where uneven pattern 14 comprised of train of grooves and/or pits thereafter to implement, e.g., Ni plating by electric plating method to form Ni plating layer 80. By peeling off this Ni plating layer 80 from glass original plate 10, the Ni plating layer 80 results in master stamper 80 including uneven pattern 81 in which uneven pattern 14 of the glass original plate 10 has been transferred.

Then, as shown in FIG. 8B, electrically conductive film (not shown) is formed by, e.g., electroless plating method is formed on the surface where uneven pattern 81 of the Ni plating layer 80, i.e., the master stamper 80 has been formed thereafter to implement, e.g., Ni plating by electroplating method to form a Ni plating layer 90. By peeling off this Ni plating layer 90 from the master stamper 80, the Ni plating layer 90 results in mother stamper 90 including uneven pattern 91 in which uneven pattern 81 of master stamper 80 has been transferred. The step of producing Ni plating layer 90, i.e., mother stamper 90 from this master stamper 80 is a step for preventing that uneven pattern 52 of disc base 51 finally produced is inverted.

Then, as shown in FIG. 8C, the uneven pattern 91 of the mother stamper 90 is transferred to, e.g., a polished acrylic original plate 100 by the 2P method with this mother stamper 90 being as matrix. Namely, first, ultraviolet hardening resin is coated onto the mother stamper 90 where the uneven pattern 91 has been formed to closely stick the acrylic original plate 100 onto this formed ultraviolet hardening resin layer 101. It is a matter of course that ultraviolet hardening resin may be coated on the acrylic original plate 100 to closely stick mother stamper 90 thereto. For example, ultraviolet rays are irradiated through the acrylic original plate 100 to harden ultraviolet hardening resin layer 101 thereafter to peel off the mother stamper 90 from the acrylic original plate 100. Thus, the uneven pattern 91 is transferred onto the ultraviolet hardening resin layer 101 so that uneven pattern 102 is formed. As a result, acrylic original plate 100 provided with ultraviolet hardening resin layer 101 including uneven pattern 102 based on the uneven pattern 91 is produced. This uneven pattern 102 consists of a flat surface portion 105, an inclined surface portion 103 and a twilled line portion 104.

Then, as shown in FIG. 8D, the surface opposite to the surface where the uneven pattern 102 has been formed of the acrylic original plate 100 is supported at the rotary stage 15 to rotate this rotary stage 15 at a constant velocity, e.g., about 60 rpm. Onto the surface of the ultraviolet hardening resin layer 101 where the uneven pattern 102 has been formed on the rotated acrylic original plate 100, ultraviolet rays are irradiated, e.g., for about four minutes and at intensity of about 10 mw/cm² by ultraviolet lamps 20 arranged under existence of oxygen or air. These ultraviolet lamps 20 are adapted so that height from the surface of the ultraviolet hardening resin layer 101 where the uneven pattern 102 has been formed on the acrylic original plate 100 on the rotary stage 15 is caused to be constant so that irradiation intensity surface density within the effective size of the surface where the uneven pattern 102 has been formed of the acrylic original plate 100 substantially becomes constant similarly to the first embodiment. In this example, as the ultraviolet lamp 20, e.g., excimer lamp having wavelength of 174 nm or low pressure mercury lamp, etc. may be used. In place of rotating acrylic original plate 60 by using rotary stage 15, ultraviolet lamp 20 may be rotated at a fixed velocity similarly to the first embodiment. The irradiation time and the irradiation intensity of ultraviolet rays are suitably selected by difference of composition of material of ultraviolet hardening resin and/or to what degree uneven pattern 102 is curved by irradiation of ultraviolet rays as described later similarly to the previously described respective embodiments.

Ultraviolet rays are irradiated from the ultraviolet lamps 20 onto the surface of the ultraviolet hardening resin layer 101 where the uneven pattern 102 has been formed on this acrylic original plate 100 under existence of oxygen or air, and ultraviolet rays are irradiated to oxygen (O₂) SO that ozone (O₃) is produced. Since this ozone (O₃) is unstable, it is decomposed into oxygen (O₂) and excited oxygen (O*) having very strong oxidation power. This excited oxygen (O*) oxidizes the surface of the ultraviolet hardening resin layer 101 which is organic matter to reform it. Thus, as shown in FIG. 9A, twilled line portion 104 of the uneven pattern 102 is chamfered and is curved, and the surface of the ultraviolet hardening resin layer 101 including flat surface portion 105 and inclined surface portion 103 constituting the uneven pattern 102 is smoothed at the same time. It is to be noted that there were cases in the first embodiment that roughness of the surface of photoresist 11 is slightly increased by irradiating ultraviolet rays onto the photoresist 11, but there is no possibility similarly to the second embodiment in the third embodiment that even if ultraviolet rays are irradiated onto the hardened ultraviolet hardening resin layer 101, roughness of the surface is increased, but the surface becomes more smooth. Accordingly, in the third embodiment, there is no necessity of irradiating infrared rays as in the case of the first embodiment. It is to be noted that, for the purpose of carrying out violet irradiation with respect to the acrylic original plate 100, violet irradiation unit may be used similarly to the first embodiment.

Then, as shown in FIG. 9B, the uneven pattern 102 of the acrylic original plate 100 is transferred onto a polished glass original plate 110 by the 2P method with this acrylic original plate 100 to which ultraviolet rays have been irradiated being as matrix. Namely, first, ultraviolet hardening resin is coated onto the acrylic original plate 100 where the uneven pattern 102 has been formed to closely stick the glass original plate 110 onto the ultraviolet hardening resin layer 111 formed by this coating. Of course, ultraviolet hardening resin may be coated onto the glass original plate 110 to closely stick the acrylic original plate 100. Ultraviolet rays are irradiated through the glass original plate 110 or the acrylic original plate 100 to harden the ultraviolet hardening resin layer 111 thereafter to peel off the acrylic original plate 100 from the ultraviolet hardening resin layer 111. Thus, the uneven pattern 102 is transferred to the ultraviolet hardening resin 111, and an original plate 120 for duplication of stamper including the ultraviolet hardening resin layer 111 where the uneven pattern 112 has been formed is produced or made. The step of transferring the uneven pattern 102 onto the glass original plate 110 by the 2P method with this acrylic original plate 100 being as matrix is a step provided for the reason why it is difficult to implement Ni plating to the surface where the uneven pattern 102 has been formed of the ultraviolet hardening resin layer 101 on the acrylic original plate 100 to produce or make stamper 40 because the surface of the ultraviolet hardening layer 101 is reformed as the result of the fact that ultraviolet rays have been irradiated. It is to be noted that acrylic original plate 100 to which ultraviolet rays have been irradiated can be used many times for the purpose of manufacturing of glass original plate 110 by the 2P method as matrix.

Then, as shown in FIG. 9C, electrically conductive film (not shown) is formed on the surface where the uneven pattern 112 has been formed on the original plate 120 for duplication of stamper by, e.g., electroless plating method thereafter to implement, e.g., Ni plating by electroplating method to form Ni plating layer 40. By peeling off this Ni plating layer 40 from the original plate 120 for duplication of stamper, the Ni plating layer 40 results in stamper 40 including uneven pattern 41 in which the uneven pattern 112 of the original plate 120 for duplication of stamper has been transferred. It is to be noted that the original plate 120 for duplication of stamper can be used many times for the purpose of manufacturing stamper 40 as matrix. Steps subsequent to ones of transferring uneven pattern 41 from this Ni plating layer 40, i.e., the stamper 40 to the disc base 51 are carried out similarly to the first embodiment.

As stated above, in the third embodiment, ultraviolet rays are irradiated onto the ultraviolet hardening resin layer 101 at a uniform intensity, whereby the surfaces of the flat surface portion 105 and the inclined surface portion 103 constituting the uneven pattern 102 are smoothed, and the twilled line portion 104 of the uneven pattern 102 can be curved. By transferring the uneven pattern 102 to the ultraviolet hardening resin layer 111 of the glass original plate 110 by using this acrylic original plate 100, it is possible to manufacture original plate 120 for duplication of stamper in which the surfaces of a flat surface portion 115 and an inclined surface portion 113 of the uneven pattern 112 have been smoothed, and a twilled line portion 114 of the uneven pattern 112 has been curved. By using this original plate 120 for duplication of stamper, the uneven pattern 112 is transferred to one surface of the disc base 51 through the stamper 40. At the optical disc base 51, it is possible to form uneven structure 52 including a flat portion 522 and an inclined surface portion 523 of which surfaces have been smoothed, and a twilled line portion 524 which has been curved. Thus, in the optical disc 50 using this disc base 51, it can be prevented that surface roughness takes place at the surface where the uneven structure 52 is formed of the disc base 51, and/or distortion in film formation takes place in recording layer 54, etc. at the twilled line portion 524 of the uneven structure 52.

As described above, even in the case where laser beams of short wavelength like, e.g., violet semiconductor laser are irradiated onto this optical disc 50 to carry out recording of information or to carry out reproduction of recorded information, noise can be reduced, and sufficient S/N can be ensured. In addition, it is possible to efficiently mass-produce, at low cost and in a short time, optical discs 50 capable of ensuring sufficient S/N in this way by the stamper 40 in which the uneven pattern 102 has been transferred through the original plate 120 for duplication of stamper from the acrylic original plate 100 to which ultraviolet rays have been irradiated.

While the present invention has been explained as above by taking several embodiments, the present invention is not limited to the above-described embodiments, but may be modified or changed in various manners. For example, while explanation has been given in the third embodiment in connection with the example where mother stamper 90 is produced from master stamper 80, plastic base may be produced from master stamper 80 by injection molding, compression molding or 2P method. It is sufficient to transfer uneven pattern to acrylic original plate 100 by the 2P method with this plastic base being as matrix. It is to be noted that in the case where uneven pattern is transferred to acrylic original plate 100 by the 2P method with molded disc which is plastic base produced by injection molding from master stamper 80 being as matrix, it is necessary to embed center hole of the molded disc.

While explanation has been given in the third embodiment in connection with the example where uneven pattern 91 is transferred to acrylic original plate 100 by the 2P method with mother stamper 90 being as matrix, plastic base in which uneven pattern 91 has been transferred by injection molding, compression molding or 2P method may be produced from mother stamper 90. It is sufficient to carry out ultraviolet irradiation with respect to this plastic base to produce stamper 40 with this plastic base to which ultraviolet irradiation has been carried out being as matrix. It is to be noted that in the case of plastic base produced from mother stamper 90 by the 2P method, it cannot be directly used as mold (matrix) for production of stamper 40 after ultraviolet rays have been irradiated, and it is therefore necessary to once transfer uneven pattern to glass original plate 110 by the 2P method. In the case where stamper 40 is produced with molded disc which is plastic base produced from mother stamper 90 by injection molding being as matrix, it is necessary to embed center hole of the molded disc. As material of this plastic base, e.g., polymethyl methacrylate and polycarbonate, etc. are preferable.

The irradiation conditions such as irradiation times and irradiation intensities, etc. of ultraviolet rays and infrared rays irradiated onto photoresist 11 in the first embodiment, ultraviolet rays irradiated onto the ultraviolet hardening resin layer 61 in the second embodiment, and ultraviolet rays irradiated onto the ultraviolet hardening resin layer 101 in the third embodiment are suitably changed by film thickness and/or material, etc. of the photoresist 11, the ultraviolet hardening resin layer 61 and the ultraviolet hardening resin layer 101.

### INDUSTRIAL APPLICABILITY

As explained above, in accordance with the present invention, it is possible to manufacture original plate of stamper for manufacturing optical recording medium in which the surface of uneven pattern corresponding to grooves, etc. recorded with respect to optical recording medium is smoothed, and twilled line portion of the uneven pattern is curved. Accordingly, uneven pattern is transferred from original plate of this stamper for manufacturing optical recording medium, thereby making it possible to obtain optical recording media which can efficiently ensure sufficient S/N ratio at the time of recording and/or reproduction of information.

1 A method of manufacturing an original plate, comprising:
exposing and developing a photoresist layer provided on a base to form uneven pattern at the photoresist layer;
irradiating ultraviolet rays to the photoresist layer where the uneven pattern has been formed; and
thereafter irradiating infrared rays onto the photoresist layer.

2 The method of manufacturing original plate as set forth in claim 1,
wherein the ultraviolet rays are irradiated onto the photoresist layer under the environment where oxygen exists.

3 The method of manufacturing original plate as set forth in claim 2,
wherein a ultraviolet rays light source which emits the ultraviolet rays is disposed in a manner spaced from the surface of the photoresist layer to irradiate ultraviolet rays to the photoresist layer.

4 The method of manufacturing original plate as set forth in claim 3,
wherein the ultraviolet rays light source and the base are relatively moved.

5 The method of manufacturing original plate as set forth in claim 1,
wherein the infrared rays are irradiated to heat the photoresist layer so that temperature becomes equal to more than softening point temperature.

6 The method of manufacturing original plate as set forth in claim 5,
wherein an infrared rays light source which emits the infrared rays is disposed in a manner spaced from the surface of the photoresist layer to irradiate infrared rays to the photoresist layer.

7 The method of manufacturing original plate as set forth in claim 6,
wherein the infrared rays light source and the base are relatively moved.

8 The method of manufacturing original plate as set forth in claim 1,
wherein plating processing is further implemented to the photoresist layer to which the infrared rays have been irradiated.

9 A method comprising:
irradiating ultraviolet rays onto a ultraviolet hardening resin layer provided on a base and adapted so that uneven pattern is formed; and
manufacturing a stamper by using the base including the ultraviolet hardening resin layer to which the ultraviolet rays have been irradiated.

10 The manufacturing method as set forth in claim 9,
wherein the ultraviolet rays are irradiated onto the ultraviolet hardening resin layer under the environment where oxygen exists.

11 The manufacturing method as set forth in claim 10,
wherein a ultraviolet rays light source which emits the ultraviolet rays is disposed in a manner spaced from the surface of the ultraviolet hardening resin layer to irradiate ultraviolet rays onto the ultraviolet hardening resin layer.

12 The manufacturing method as set forth in claim 11,
wherein the ultraviolet rays light source and the base are relatively moved.

13 The manufacturing method as set forth in claim 9, comprising:
allowing a further base a further ultraviolet hardening resin layer is provided to be in closely contact with the ultraviolet hardening resin layer to which the ultraviolet rays have been irradiated; irradiating ultraviolet rays to harden the further ultraviolet hardening resin layer to transfer the uneven pattern formed at the ultraviolet hardening resin layer to the further ultraviolet hardening resin layer; and implementing plating processing to the further ultraviolet hardening resin layer to which the uneven pattern has been transferred to form a stamper.

14 A manufacturing method comprising:
exposing and developing a photoresist layer provided on an original plate to form uneven pattern at the photoresist layer;
implementing plating processing to the photoresist layer where the uneven pattern has been formed to form master stamper;
irradiating ultraviolet rays to a base where a ultraviolet hardening resin layer is provided and the master stamper in the state where they are caused to be in closely contact with each other to transfer uneven pattern of the master stamper to the ultraviolet hardening resin layer;
thereafter further irradiating ultraviolet rays to the ultraviolet hardening resin layer; and
manufacturing a stamper by using the base including the ultraviolet hardening resin layer to which the ultraviolet rays have been irradiated.

15 The manufacturing method as set forth in claim 14,
wherein the ultraviolet rays are irradiated onto the ultraviolet hardening resin under the environment where oxygen exists.

16 The manufacturing method as set forth in claim 15,
wherein a ultraviolet rays light source which emits the ultraviolet rays is disposed in a manner spaced from the surface of the ultraviolet hardening resin layer to irradiate ultraviolet rays to the ultraviolet hardening resin layer.

17 The manufacturing method as set forth in claim 16,
wherein the ultraviolet rays light source and the base are relatively moved.

18 The manufacturing method as set forth in claim 14, comprising:
allowing a further base where a further ultraviolet hardening resin layer is provided to be in closely contact with the ultraviolet hardening resin layer to which the ultraviolet rays have been irradiated; irradiating ultraviolet rays to harden the further ultraviolet hardening resin layer to transfer the uneven pattern formed at the ultraviolet hardening resin layer to the further ultraviolet hardening resin layer; and implementing plating processing to the further ultraviolet hardening resin layer to which the uneven pattern has been transferred to form a stamper.
